(19) United States
(12) Patent Application Publication
(10) Pub. No.: US 2012/0277750 A1
(43) Pub. Date: Nov. 1, 2012

(54) ADJUSTABLE SUCTION SHAVER

(75) Inventors: WERNER BUTSCH, DUERBHEIM (DE); ROLAND-ALOIS HOEGERLE, TUTTLINGEN (DE)

(73) Assignee: AESCULAP AG, TUTTLINGEN (DE)

(21) Appl. No.: 13/417,816

(22) Filed: Mar. 12, 2012

(30) Foreign Application Priority Data
Mar. 14, 2011 (DE) 10 2011 001 265.6

(51) Int. Cl. A61B 17/56

(52) U.S. Cl. 606/84

(57) ABSTRACT

A medical tissue-abrading instrument, for instance a shaver, includes an instrument handpiece at which manipulators for actuating an abrasion means, for instance a cutting or grinding head, are arranged and in which a fluid passage adjustable in cross-section by means of a valve is formed for sucking off abraded tissue and/or for supplying scavenging fluid. The valve is a manually operable slider valve. The valve piston of the valve includes a control edge having an edge shape which causes a change of the passage cross-section at a particular ratio, preferably linearly, during continuous actuation of the valve.

### RELATED APPLICATIONS

This application claims the benefit of priority of German Application No. DE 10 2011 001 265.6, filed Mar. 14, 2011, the content of which is incorporated by reference in its entirety and for all purposes.

### FIELD

The present invention relates to a medical instrument comprising a suction device and especially a suction shaver having a flexible or adjustable suction capacity for arthroscopic surgery.

### BACKGROUND

A shaver is a medical instrument which is employed especially in arthroscopy for abrading and removing fine soft tissue or cartilage. It comprises substantially a tube/tubular shank, preferably made of stainless steel, the distal end of which is spared toward a so-called window. In said window an outwardly exposed knife in the form of a rotor head is arranged which can be put in rotation by a driving shaft supported in the tubular shank. An electric motor provided in the handpiece of the instrument and adapted to be activated by a pedal or a finger key makes the knife rotate to the left or to the right, with a reciprocating movement also being possible. Abraded tissue material such as chips and tissue residues are sucked through the internal passage in the stainless steel shank.

A shaver of this generic type is known from the state of the art, for instance according to U.S. Pat. No. 6,436,067.

Said shaver includes at the handpiece next to the actuating keys/buttons required to activate the electric motor a rotary valve interposed in a suction passage in the handpiece between the stainless steel tube mounted to the handpiece and a suction connection. Said rotary valve consists of a bolt rotatably supported in the handpiece in which a cross bore is formed and to which a manipulator in the form of a pivoting lever is fixed. The cross bore thus constitutes a conduit in the suction passage which is adapted to be opened and closed. I.e. by turning the bolt traversing the suction passage the passage is either blocked or opened, wherein also intermediate positions of the bolt are possible for partially opening the suction passage. For this purpose, the pivoting lever is supported by a detent mechanism on the handpiece so that a number of detent positions selected according to said detent mechanism can be adopted. In this way, the rotary valve can be opened and closed in a quasi continuous manner so as to adjust an appropriate volume flow of sucked material.

The rotary valve and the lever to be actuated are placed at a position of the handpiece which is radially facing away from the arrangement of the actuating keys for the motor. Practically speaking, the actuating means/keys for the motor are arranged so that they can be reached by the thumb of a hand enclosing the handpiece as prescribed (viz. on the upper side of the handpiece), whereas the pivoting lever is supported on the handpiece at the outside of the latter facing away therefrom (viz. on the lower side of the handpiece). The pivot axis of the lever extends transversely to the direction of extension of the handpiece so that the lever can be shifted in the longitudinal direction of the instrument.

It has turned out that, although the suction volume of the shaver can be adjusted in a comparatively sensitive manner with this design, the actuation of the pivoting lever is not ergonomic and consequently difficult during a surgical intervention. Especially when actuating the pivoting lever in a surgical intervention phase, a second hand is required which entails considerable drawbacks during handling the instrument, such as unintended deflections of the instrument, possibly temporary releasing of secondary instruments etc.

Also in practical application of the known shaver the pivoting lever has proven to be unreliable to the effect that it does not permit reproducible adjustment of the opening cross-section in the suction passage, as the lever necessarily protruding from the handpiece can easily actuated inadvertently, especially also because it is arranged at the lower side of the handpiece with respect to the surgeon and therefore is not (easily) seen by the surgeon.

### SUMMARY

In view of this situation, it is an object of the present invention to provide a medical (surgical) instrument comprising a suction device and especially a generic shaver that exhibits a functionality improved vis-á-vis the known state of the art. It is an object in this respect to increase the functional reliability of the instrument. It is another object to optimize the manageability and, where appropriate, the ergonomics of the instrument and, in particular, of the handpiece thereof.

The afore-mentioned object is achieved by a medical instrument (especially a shaver) having flexible or adjustable suction preferably for minimal-invasive surgery comprising the features of claim 1. Advantageous embodiments of the invention are the subject matter of the subclaims.

Accordingly, the invention consists of providing a medical, in particular tissue-abrading instrument comprising an instrument handpiece at which manipulators for actuating an abrasion means of the instrument are arranged. In the instrument handpiece a fluid passage adjustable or variable in cross-section by means of a valve or a throttle for discharging or sucking abraded tissue and/or for supplying scavenging fluid is formed.

In accordance with an aspect of the invention, the valve or the throttle is a manually or electrically operable slider valve. The slider valve allows to precisely and easily vary the efficient flow cross-section at this position of the fluid passage and thus to adjust the flow volume.

Another aspect of the invention dependent on or independent of the foregoing aspect provides for actuating the valve or the adjustable throttle (of any design) a manipulator movably supported on the instrument handpiece which is either mechanically coupled to the valve or the adjusting throttle or actuates an electric drive, solenoid, piezo drive or similar regulating mechanism for adjusting the valve (or the adjusting throttle). It is advantageous in this respect, however, when the valve in the form of a slider valve includes a valve piston (mechanically) coupled to the manipulator which is preferably supported to be oriented in parallel to the valve piston slidably on the instrument handle or handpiece.

Due to the slidable support of the manipulator on the handpiece, in contrast to the known state of the art no lever protrudes from the handpiece even in intermediate positions (partly opened valve) so that there is a considerably lower risk of inadvertent adjustment. Consequently the functional reliability is increased.

It is further advantageous when the manipulator includes a sliding button guided along a gate that is preferably formed slit-shaped in a housing of the handpiece. This design enables the manipulator for actuating the valve in accordance with any one of the foregoing aspects to be integrated quasi in the handpiece surface without or with only little projection, which further improves the functional reliability of the manipulator to prevent inadvertent actuation.

Especially in the case of a slider valve design the fluid passage can include a supply passage portion intersecting the sliding path of the valve piston further preferably at right angles (or at any angle). It is of advantage in this case when said passage portion extends toward the housing wall of the instrument handpiece which is outwardly gripped by a surgeon's hand in the case of operation. This permits placing the slider valve or the valve piston directly below the housing of the instrument handpiece (below the housing wall) and coupling the manipulator immediately to the valve piston, for instance by a pin, as the displacing path of the valve piston now can extend substantially in parallel to the housing wall.

In such instrument handpiece basically further functional passages or conduits for coupling the other manipulators to functional units of the instrument are laid. Therefore, another aspect of the invention, which is possibly independent or applicable in combination with the foregoing aspects, provides that the supply passage portion intersecting the valve consists of two passage arms connected in parallel that (in the case of a slider valve viewed in the sliding direction of the valve piston) converge at a preferably obtuse angle and thus end into the (slider) valve from two substantially opposing radial sides. By at least partially forming two passage arms the individual cross-sections thereof can be optionally reduced (without the total flow cross-section being reduced) so that the passage arms can be laid more easily between the individual conduits and functional passages or integrated in the housing.

It is advantageous in this context when in the case of a slider valve the two passage arms are arranged, with respect to the sliding path of the valve piston, level with each other or are offset from each other in the sliding direction. The two passage arms are varied in cross-section in parallel (simultaneously) in the first case and serially (staggered in time/partly overlapping) in the second case.

After all, it is provided according to a further aspect of the invention preferably independent of or applicable in combination with at least one of the foregoing aspects that a valve piston of the valve (possibly of any design) exhibits at least one control edge which is configured with respect to the cross-sectional shape of the passage cross-section so that the passage cross-section varies preferably linearly at a (pre)defined relation when the valve is continuously actuated. The sensed handling or haptics of the instrument can be improved by appropriately associating the degree of actuating the manipulator with the closing/opening behavior of the valve in accordance with the shape of the control edge (and also the cross-sectional shape of the supply passages).

In addition, the handling is improved according to another, possibly independent or combined aspect by the fact that the manipulator for actuating the valve is arranged at the same angular position as the manipulators for actuating the abrading means and is spaced apart from the latter in the longitudinal direction of the handpiece. In this way, all afore-mentioned manipulators can be actuated by the same finger (thumb) without changing the grip. It is superfluous in any case to use one's second hand.

### BRIEF DESCRIPTION OF THE DRAWINGS

Hereinafter the invention shall be illustrated by way of preferred embodiments with reference to the accompanying figures.

FIG. 1 shows the side view of a medical tissue-abrading instrument, preferably in the form of a shaver, according to a first preferred embodiment of the invention,

FIG. 2 shows a top view of the instrument according to FIG. 1,

FIG. 3 shows a longitudinal section of the instrument according to FIG. 1 including an opened suction means,

FIG. 4 shows a cross-section of the instrument along the section B-B according to FIG. 1,

FIG. 5 shows a cross-section of the instrument along the section C-C according to FIG. 1,

FIG. 6 shows a longitudinal section of the instrument according to FIG. 1 including a closed suction means, and

FIGS. 7a-7c show the schematic representation of a valve for actuating a suction means of a medical tissue-abrading instrument, preferably in the form of a shaver, according to a second preferred embodiment of the invention at an opened valve position and a closed valve position.

### DETAILED DESCRIPTION OF PREFERRED EMBODIMENTS

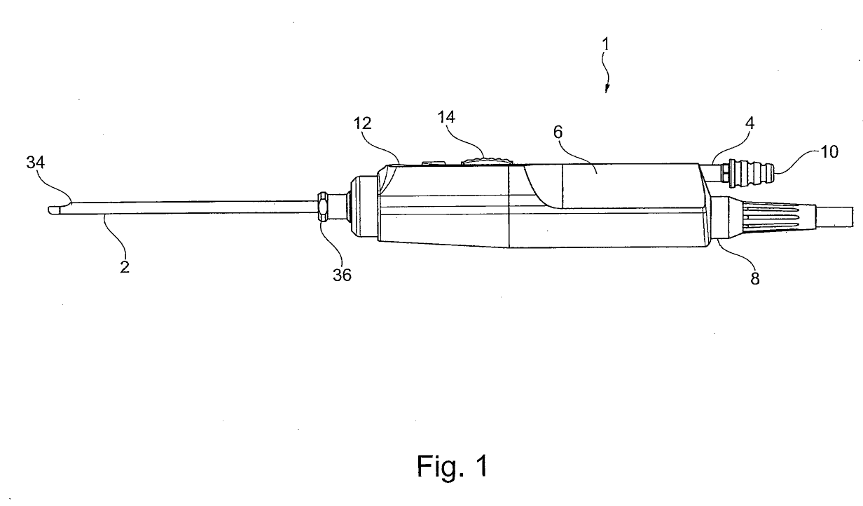

FIGS. 1 to 3 illustrate a medical tissue-abrading instrument 1, presently shown in the form of a so-called shaver, as it is used, for instance, in minimal invasive surgery, preferably in arthroscopy.

Basically an instrument 1 of this type substantially comprises three functional groups, an abrasion means 2, a scavenging and/or suction means 4 and a multi-functional instrument handpiece 6 including electric 8 and fluid connections 10 as well as manipulators 12, 14 for actuating the abrasion 2 and scavenging/suction means 4.

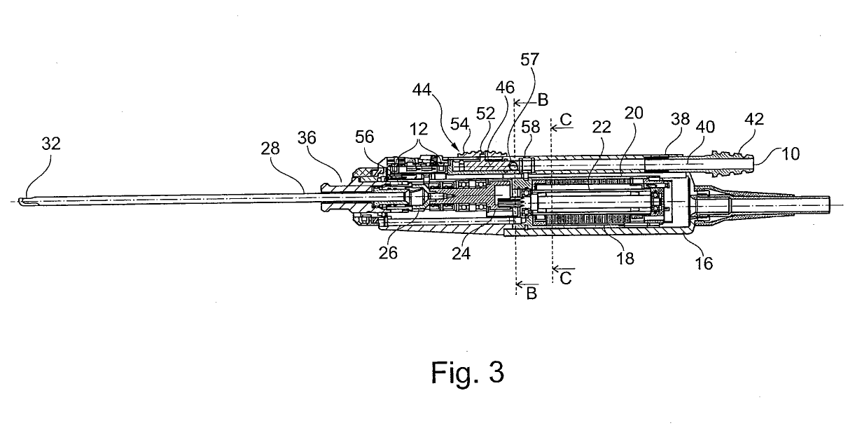

Practically speaking, the instrument handpiece 6 according to FIG. 3 is made of a multi-part, where appropriate, and substantially cylindrical housing shell 16 at the proximal end face of which the electric connection 8 as well as the fluid connection 10 is formed and arranged, respectively. Alternatively, also a combined connection may be provided to which a hose package consisting of electric conductors and a fluid hose can be connected.

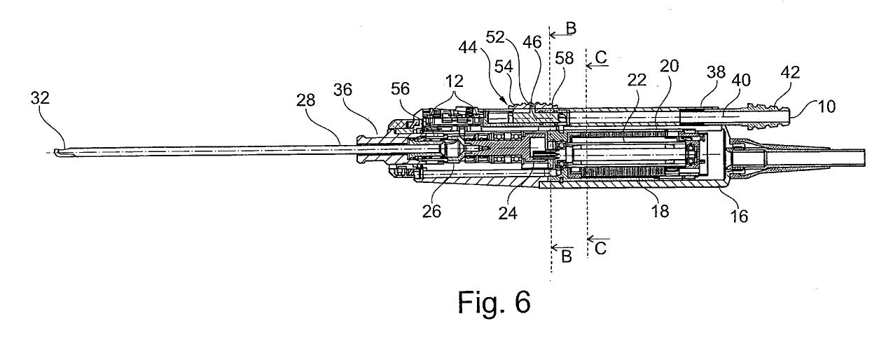

Inside the instrument handpiece 6 according to FIG. 3 or 6 an electric motor 18 is accommodated consisting of a stator 20 fixed in the housing 16 and a rotor 22 supported in said stator. For example, the electric motor 18 can be an encapsulated motor cartridge fitted into the housing 16. A driven shaft of the motor 18 is coupled to a transmission 24 which is equally arranged in the housing 16. The motor 18 and the transmission 24 in the present case constitute two separate units so to be able, as required, to realize or combine different transmission ratios and torques/speeds according to the modular design. It is also possible, however, to form the motor 18 and the transmission 24 inside the cartridge (quasi in one piece).

A rotating rod 28 which is supported to rotate in a tubular shank 30 mounted or screwed onto the distal end face of the instrument handpiece 6 is coupled to the transmission 24 via a rotating joint 26. At the distal end of the rotating rod 28 a cutting, milling or grinding head 32 is arranged (formed) which freely projects from a radial cut-out (window) 34 at the distal end portion of the tube 30.

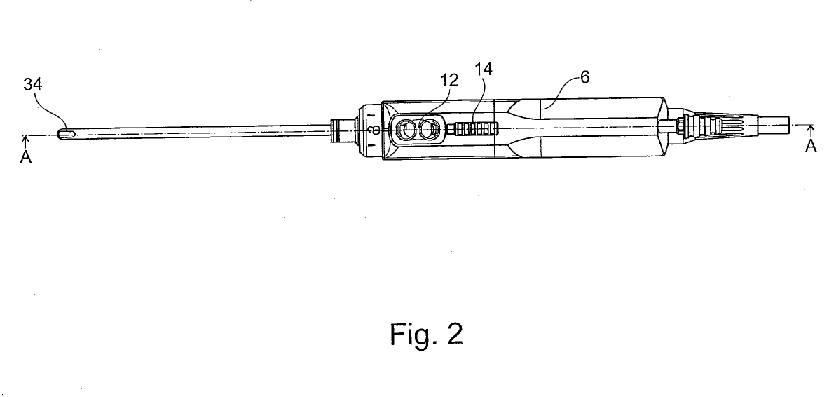

As can be taken from FIGS. 1, 2, in the present case the tube (tubular shank) 30 is fixed to the instrument handpiece 6 by means of a sleeve nut 36. It is also possible, however, to provide a bayonet or spring lock. Furthermore, the rotating rod 28 is guided within the tube 30 (preferably made of a metal alloy) so that an annular gap around the rotating rod 28 is formed through which scavenging fluid can be conveyed from the instrument handpiece 6 to the window 34 and/or abraded material can be sucked through the window 34 toward the instrument handpiece 6.

In parallel to the motor cartridge 18 the scavenging/sucking means 4 is formed in the instrument handpiece 6. It consists of a first connecting passage 38 extending in the longitudinal direction of the instrument handpiece (in parallel to the motor cartridge 18) which ends directly into the fluid connection 10. Preferably an external insert tube 40 is inserted (in a sealing manner) in the first connecting passage 38 formed in the handpiece housing 16, the insert tube (axially) projecting from the first connecting passage 38 or from the proximal end face of the instrument handpiece 6 and at its free end being in the form of a widening plug 42 for pulling on a hose (not shown in detail). Alternatively, it is also possible to screw a sleeve-like hose connection into the connecting passage.

A valve 44 (operable continuously or in several steps) for selectively opening and closing the first connecting passage 38 continuously/stepwise (by detents) is inserted in the first connecting passage 38 to be axially adjacent to the (optional) insert tube 40. Said valve 44 consists of a valve piston 46 guided axially movably in the first connecting passage 38 and forming a control edge at its end face facing the fluid connection 10. In the area of the valve piston 46 two second supply passages 48 extending substantially at right angles with the first connecting passage 38 and at an obtuse angle with each other according to FIG. 5 end into the first connecting passage 38. The openings of the two supply passages 48 radially opposing with respect to the connecting passage 38 are positioned at the first connecting passage 38 so that they are completely opened when the valve piston 46 takes a first retracted end position (to the left according to FIG. 3) and are completely closed when the valve piston 46 takes a second advanced end position (to the right according to FIG. 3). Moreover, the orifices are arranged at the same level with respect to the displacing direction of the valve piston 46 so that they can be opened/closed simultaneously and uniformly by the valve piston 46. They can as well be offset in the displacing direction of the valve piston 46, however.

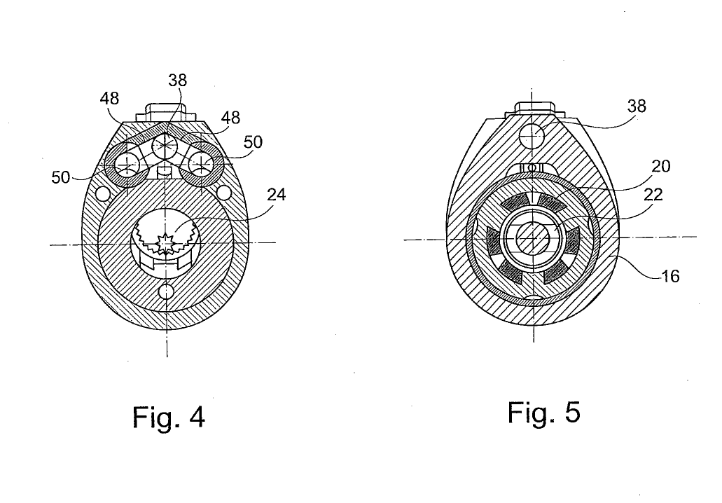

In accordance with FIGS. 4 and 5, each of the second supply passages 48 has a somewhat smaller diameter (they can also have a substantially equal diameter) and is positioned so that they bypass the motor cartridge 18 and the transmission 24 at the peripheral side. In this way the two supply passages 48 create a fluid communication between the annular gap formed in the tube (tubular shank) 30 and the first connecting passage 38 via two further (third) axial passages 50 (cf. FIG. 4) extending in the instrument handpiece 6 along the transmission 24 and/or the motor cartridge 18 and connecting the two supply passages 48 to the annular gap.

According to FIG. 3, at the valve piston 46 a driving lug or pin 52 is formed or inserted which extends radially from the valve piston 46 and outwardly penetrates a longitudinal slit (not shown in detail) in the handpiece housing 16. Said longitudinal slit constitutes a guiding gate extending along the connecting passage 38 and defining the two final positions of the valve piston 46. Moreover, the longitudinal slit inhibits a rotation of the valve piston 46. The latter may be important in so far as the control edge of the valve piston 46 may have a predetermined shape which causes, for instance, linear closing/opening of the two supply passages 48 upon continuous (uniform) displacing motion of the piston 46.

A sliding button 54 constituting the manipulator 14 for manually operating the valve piston 46 is mounted on the driving lug 52.

In this context it is referred to the fact that this design can also be modified. For example, the sliding button 54 can be replaced with a rotatably supported operating roll or gearwheel which is in tooth mesh with the valve piston and, consequently, transforms a rotation of the manipulator to the translatory motion of the valve piston. As a further alternative, also the valve piston could be supported in a rotatable instead of a displaceable manner, wherein it would extend transversely to the connecting passage and intersect the same in this case. Then a central cross bore would be formed in the valve piston such that at a particular angle of rotation the connecting passage would be open directly downstream of the orifices of the supply passages and would be closed in the case of a rotation by 90° from that. For actuating said rotary valve again the sliding button could be used as manipulator which in this case would be connected to the rotary valve (rotary piston) via an eccentric rod so as to transform a translatory motion to the rotation of the rotary piston.

It can further be inferred from FIGS. 1 and 2 that at the instrument handpiece 6 the further manipulators 12 are provided in the form of push buttons for actuating the abrasion means 2/the motor 18. They are arranged, according to FIG. 2, in the direction of the tube (tubular shank) 30, i.e. ahead of the manipulator 14 of the suction means 4 in the same angular position. Practically speaking, the connecting passage 38 according to FIG. 3 ends at the distal (front) third of the instrument handpiece 6, thereby a free space remaining in this portion axially ahead of the connecting passage 38 radially offset from the transmission 24 in the instrument handpiece 6. Electric switching elements 56 which are manually operable by the push buttons 12 and which are connected to the motor cartridge 18 via electric conductors (not shown in detail in the Figures) are inserted in this space.

The push buttons or keys for operating the motor 18 provided with the reference numerals 12 can also be replaced or supplemented with foot-operated switches (not shown in detail) in practice. I.e. either the keys 12 are completely dropped or they are put out of operation by a cable package connected to the instrument handpiece, namely when the foot-operated switch(es) is/are equally connected thereto or they remain basically in operation, wherein the foot-operated switch connected thereto is also in operation. These variants provide different advantages:

If the instrument handpiece is designed without the push buttons 12 so that only an operation by the foot-operated switch via the electric cable is possible, the handpiece design is simplified. If the push buttons 12 are retained at the instrument handpiece, wherein they can be supplemented by a foot-operated switch or they can be replaced as to their function, the surgeon can choose by which manipulator (hand and/or foot manipulator) he/she intends to activate the motor 18. Moreover, the instrument then can be used universally in that it is optionally operable only by hand or by hand and foot or only by foot.

On principle, instruments of this generic type must not constitute any risk of contamination of the site of the operation with the sucked tissue materials and fluids. Therefore, the valve piston 46 is supported to be sealed in the connecting passage 38 both in the sliding and in the rotating type. In accordance with FIG. 3, for this purpose in the longitudinal portion between the control edge and the driving pin 52 a first annular packing 57 is inserted in a peripheral housing groove for sealing the valve piston 46 toward the guiding slit. Moreover, at a longitudinal portion immediately downstream with respect to the two orifices likewise a packing ring 58 is inserted in a peripheral groove at the housing side which additionally seals the valve piston 46 at its second closed end position according to FIG. 6a.

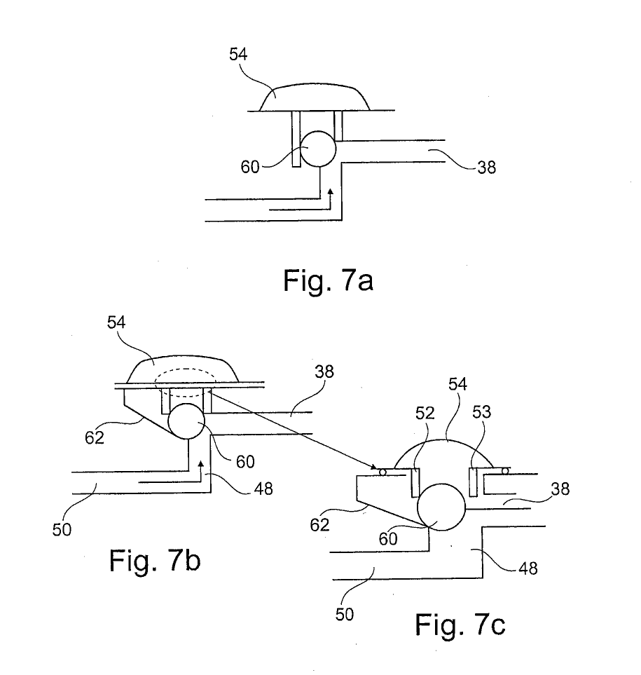

In FIGS. 7a to 7c a second preferred embodiment for a medical instrument 1 according to the invention is represented in sections, wherein hereinafter only the features different from the first embodiment shall be discussed.

As can be seen from the schematic representations of FIGS. 7a to 7c, the valve piston 46 of the slider valve 44 according to FIG. 3 can be replaced with a float 60. In this case, the manipulator in the form of a sliding button 54 includes two driving pins 52, 53 guided through the longitudinal slit and offset in a fork-like manner from each other in the sliding direction. The float 60 is held between the driving pins 52, 53 with little play.

The float 60 is supported on an inclined plane 62 within the connecting passage 38 which plane is directly aligned with the orifice(s) of the supply passages 48. When the manipulator is displaced in parallel to the connecting passage 38, the float 60 is rolled along the inclined plane 62 and thus moved toward or away from the orifices.

It is finally referred to the fact that in all foregoing embodiments and modifications the valve 44 is continuously operable mechanically via the manipulator 54. On principle, it is also possible, however, to provide the valve 44 with an actuator such as a solenoid or a piezo element and to design the manipulator 54 as potentiometer. Depending on the position (and intermediate positions) of the potentiometer, the actuator is consequently excited so as to move the valve piston 46 to a closing/opening position or intermediate positions corresponding to the position of the potentiometer.

By designing the manipulator in the form of a sliding button or a turning roll according to an aspect of the invention it is possible to countersink the manipulator quasi into the housing of the instrument handpiece (by radially resetting the guiding gate with respect to the handpiece periphery, for instance) so that the manipulator does not project or only slightly projects radially from the periphery of the handpiece. Thus inadvertent actuation of the manipulator is prevented. In the case of a slidable valve piston as a different/additional aspect of the invention, a motion transforming means between the manipulator and the valve is dropped so that the design can be designed in a simple manner. Furthermore, the slidable valve piston offers better possibilities in designing the control edge.

As a consequence, a medical tissue-abrading instrument, for instance a shaver, is disclosed comprising an instrument handpiece at which manipulators for operating an abrasion means, for instance a cutting, milling or grinding head, are arranged and in which a fluid passage adjustable in cross-section by means of a valve is formed for sucking off abraded tissue and/or for supplying scavenging fluid. In accordance with an aspect of the invention, the valve is a manually or electrically operable slider valve. According to an additional or alternative aspect, the manipulator of the valve is a sliding head or a turning roll. According to another additional or alternative aspect, the slidable valve piston includes a control edge having an edge shape that causes a change of the passage cross-section at a particular ratio, preferably linearly, during continuous actuation of the valve.

1. A medical tissue-abrading instrument comprising an instrument handpiece at which manipulators for operating an abrasion means are arranged and in which a fluid passage adjustable in cross-section by means of a valve for sucking off abraded tissue and/or for supplying scavenging fluid is formed, the valve being a manually or electrically operable slider valve.

2. The medical instrument according to claim 1 comprising an individual manipulator for actuating the valve which is slidably supported at the instrument handpiece.

3. The medical instrument according to claim 2, wherein the valve includes a valve piston which is mechanically coupled to the individual manipulator.

4. The medical instrument according to claim 2, wherein the individual manipulator includes a sliding button being guided along a gate which is formed preferably slit-like in a housing of the instrument handpiece.

5. The medical instrument according to claim 3, wherein the fluid passage includes a supply passage portion that intersects the sliding path of the valve piston preferably at right angles.

6. The medical instrument according to claim 5, wherein the valve piston includes at least one control edge which completely closes, completely releases and/or partly releases the supply passage portion in response to the sliding position.

7. The medical instrument according to claim 5, wherein the supply passage portion consists of two passage arms connected in parallel which converge at a preferably obtuse angle viewed in the sliding direction of the valve piston and thus end into the valve from two substantially opposing radial sides.

8. The medical instrument according to claim 7, wherein the two passage arms are arranged at the same level or are offset from each other in the sliding direction with respect to the sliding path of the valve piston.

9. The medical instrument according to claim 3, wherein a radially projecting driving pin to which the manipulator is mounted is arranged or formed at the valve piston.

10. The medical instrument according to claim 3 comprising a sealing member positioned so that it encloses the valve piston in a sealing manner against the manipulator also at its final position completely releasing the fluid passage.

11. The medical instrument according to claim 1, wherein the valve comprises a valve piston, and the valve piston includes at least one control edge configured so that the passage cross-section varies at a particular ratio, preferably linearly, during continuous actuation of the valve.

12. The medical instrument according to claim 1, wherein the medical instrument is a shaver that includes a suction device adapted to adjust a volume flow.

13. The medical instrument according to claim 1, wherein, for actuating the valve, the individual manipulator is arranged at the angular position of the manipulators for actuating the abrasion means and is spaced apart from the same in the longitudinal direction of the instrument handpiece.
